(19) United States
(12) Patent Application Publication
(10) Pub. No.: US 2022/0104548 A1
(43) Pub. Date: Apr. 7, 2022

(54) AEROSOL DELIVERY DEVICES

(71) Applicant: Cambridge Consultants Limited, Cambridge, Cambridgeshire (GB)

(72) Inventors: Christopher James ROSSER, Cambridge (GB); Simon James SMITH, Hertford (GB); Joanne Louise CHANNON, Cambridge (GB); Sophia Faye GODFREY, Cambridge (GB)

(73) Assignee: Cambridge Consultants Limited, Cambridge, Cambridgeshire (GB)

(21) Appl. No.: 17/426,676

(22) PCT Filed: Jan. 30, 2020
(86) PCT No.: PCT/GB2020/050218

§ 371 Date: Jul. 29, 2021

(30) Foreign Application Priority Data
Jan. 30, 2019 (GB) 1901271.5

(51) Int. Cl. A24F 40/44; A24F 40/46; A24F 40/70; A24F 40/53; A24F 40/10

(52) U.S. Cl. CPC A24F 40/44; A24F 40/46; A24F 40/10; A24F 40/53; A24F 40/70

(57) ABSTRACT

The invention provides an electronic inhaler for the generation of a condensation aerosol from a liquid comprising: a reservoir (114) adapted to contain a liquid for aerosolization; a heating element (108) having a pre-defined shape; a wicking element (112) formed from fused beads of an amorphous solid so as to at least partially conform to the shape of the heating element (108), thereby providing a porous structure adapted to transport liquid from the reservoir (114) to the heating element (108) such that the heating element (108) is operable to heat the wicking element (112) thereby vaporising at least a portion of the liquid transported from the reservoir (114) by the wicking element (112); and an airflow path (115) to allow the flow of a condensation aerosol formed by said vaporised liquid. The invention further provides a method for the manufacture of a heater-wick element adapted for use in the electronic inhaler.

The present invention generally relates to aerosol delivery devices and methods for the production of condensation aerosols which carry active components for inhalation. In particular, it relates to electronic inhalers.

Devices which generate a condensation aerosol for inhalation are becoming increasingly popular. Through the use of heat to vaporise a liquid formulation these produce a condensation aerosol which contains one or more active ingredients, such as active drugs, medicines, flavouring agents, etc. Aerosol delivery devices include inhalers used by patients to deliver medication in the form of an aerosol to the body via the lungs. Other examples include electronic smoking devices such as electronic cigarettes (“e-cigarettes”). E-cigarettes simulate the act of tobacco smoking by producing an inhaled aerosol (commonly referred to as a “vapour”) which has the appearance, flavour and feel of tobacco smoke. Compared to tobacco smoking, e-cigarettes provide a safer smoking experience by eliminating the combustion process that occurs when tobacco is smoked which gives rise to toxins and carcinogens.

Aerosol delivery devices typically contain a reservoir which holds the liquid containing one or more active ingredients for aerosolization, a suitable wicking structure, and a heating element. By capillary action the wicking structure draws liquid from the reservoir which, on heating, is vaporised. Subsequent cooling of the vapour provides a condensation aerosol which carries the desired active ingredient(s) for inhalation.

Wicking elements presently used in aerosol delivery devices (e.g. spun silica glass, porous ceramics, cotton, and fused metal balls) suffer from various problems. These can be difficult to make and, in many cases, do not meet the necessary quality standards for a medical device in view of the nature of the materials employed to produce the wick and/or the methods used in their manufacture.

The manufacturing process for a silica glass wick is complex and includes an acid leaching stage. This is required for good liquid transport, however, it damages the mechanical integrity of the wick causing problems in manufacture and creating the potential for particulate generation when in use. Cotton wicks may be associated with significant health risks if overheated. They can also cause long term health problems, for example these can cause conditions such as Byssinosis (“Cotton Pickers Lung”). Cotton is also a slow wicking material which means there is a long delay between initial filling of the device with the liquid for vaporisation and first use.

When ceramics are processed to achieve sufficient porosity for use as a ceramic wick, these materials can become brittle and can fracture under stress. This produces particulates (crystalline dust) during use which gives rise to safety concerns when inhaled, for example Silicosis. The brittle nature of ceramics further limits their use to specific wick geometries.

Fused metal balls must be provided with an electrically insulated coating when used as a wicking structure in combination with an electrical heating element. Their thermal mass also reduces the efficiency of the vaporiser. When metal balls are used there is also a risk of thermally cycling the liquid for vaporisation thereby increasing the tendency for the production of degradation products. Users also report an unpleasant metal taste when using vaporisers having a metal wick.

When using silica glass or cotton with an electrical heating element, these require that the heating element is wound onto the wick itself. Not only is this a complex process in terms of manufacturing (since this often must be done by hand rather than by machine), but it can give rise to the production of particulates (and hence safety concerns when in use) and there is inevitably a variability in the degree of coil tension which can affect the performance of the device.

For at least some of these reasons, existing wicking materials and manufacturing methods are not medically approved. There thus exists a need for alternative wicking materials and methods for their manufacture which can provide the desired combination of material properties, for example wicking ability, structural integrity, safety, insulation, and liquid hold-up.

When viewed from a first aspect the present invention provides an aerosol delivery device (e.g. an electronic inhaler) for the generation of a condensation aerosol from a liquid comprising:
a reservoir adapted to contain a liquid for aerosolization;
a heating element having a pre-defined shape;
a wicking element formed from fused beads of an amorphous solid so as to at least partially conform to the shape of the heating element, thereby providing a porous structure adapted to transport liquid from the reservoir to the heating element such that the heating element is operable to heat the wicking element thereby vaporising at least a portion of the liquid transported from the reservoir by the wicking element; and
an airflow path to allow the flow of a condensation aerosol formed by said vaporised liquid.

Thus it will be seen that the inventors have appreciated that improvements can be made in respect of known wicks for use in aerosol delivery devices and propose the use of a wick formed to conform to the shape of the heating element in which the wicking element is constructed from fused amorphous beads, such as fused glass beads. These produce the right physical and material properties and also allow manufacture of the device under conditions which lend themselves to medical manufacture.

By forming the wicking element so that it conforms to the shape of the heating element, a more intimate engagement between them may be achieved and manufacturing convenience may be increased. Those skilled in the art will appreciate that the positive mechanical engagement which may advantageously be achieved by conforming the wick to the heating element means that they may be considered as a single element (a “heater-wick” element) which, at least in some embodiments, is integrally formed.

Viewed from another aspect therefore the present invention provides an aerosol delivery device (e.g. an electronic inhaler) for the generation of a condensation aerosol from a liquid comprising:
a reservoir adapted to contain a liquid for aerosolization;
a heating element;
a wicking element formed from fused beads of an amorphous solid and integrally formed with the heating element, thereby providing a porous structure adapted to transport liquid from the reservoir to the heating element such that the heating element is operable to heat the wicking element thereby vaporising at least a portion of the liquid transported from the reservoir by the wicking element; and an airflow path to allow the flow of a condensation aerosol formed by said vaporised liquid.

Viewed from another aspect the invention provides a method of forming a condensation aerosol from a liquid, the method comprising the following steps:
providing an aerosol delivery device (e.g. an electronic inhaler) as herein described;
activating a power source provided within the device to cause a flow of electrical current from the power source to the heating element;
transporting a liquid for aerosolization from a reservoir which contains said liquid; and
heating the liquid to form a vapour which, on subsequent cooling, provides a condensation aerosol.

As will be understood, the devices and methods herein described are intended to provide one or more active components in an inhalable form. It is generally envisaged that these will be produced in the form of a condensation aerosol. The term “condensation aerosol” refers to an aerosol which has been formed by the vaporisation of a liquid and subsequent cooling of the vapour such that the vapour condenses to form particles. However, the precise physical form of the particles is not intended to be limiting and, dependent on the nature of the liquid and the conditions under which it is vaporised, it may exist in a vapour or an aerosol state, or a combination thereof.

The invention provides an aerosol delivery device comprising a number of components provided within a suitable housing. Typically, each of the components of the device herein described will be contained within the housing but that is not essential. Thus in a set of embodiments, one or more of these components may be provided external to the housing, for example these may be removably attached to the exterior of the housing.

Typically, the housing may comprise an elongated body which may be substantially cylindrical in shape. For example, in certain embodiments it may resemble a cigarette or a cigar. The housing may be a unitary body, or it may be formed from two or more pieces which are separable. For example, one part may contain one or more components which are reusable and which can be removably attached to a second part which contains one or more disposable components.

Small beads of an amorphous solid are used to form the wicking element. These are fused together to form an interconnecting network of voids (“pores”) which enables the transport of liquid from the reservoir to the heating element. The Applicant has recognised that the fusing of beads of an amorphous solid to produce the wicking element provides a number of advantages. Unlike conventional wicks, such as those made from ceramic materials or cotton, these do not generate particulates during manufacture or in use thereby improving their safety. The process of fusing the beads to produce the wicking structure also allows for these to be closely packed within and/or around a suitable heating element. This process means that at its interface with the wicking element it is, at least to some extent, ‘moulded’ to the shape of the heating element to provide an “integrally formed” heater-wick element which can be made to any desired shape and configuration and which avoids the need to wind a heating element onto a wick once the wick has been formed. This enables manufacture of the combined heater-wick element by machine rather than by hand, providing less variability in the process and avoiding problems associated with wick tension which may affect the performance of the device.

The heating element may take any of a number of appropriate configurations. Some examples include a lattice, mesh or cellular which could be formed into a tube, cylinder, prism or any other suitable shape. In a set of preferred embodiments the heating element comprises wire, further preferably formed into an open helical coil shape. This is convenient to make and allows the characteristics of the heating element to be varied by simply varying the diameter and/or pitch of the helix or the gauge or material of the wire. In such embodiments the wicking element can therefore be formed by fusing the amorphous beads so that the resultant structure has an undulating shape which substantially conforms to the contours of the coil. The coil could have a constant diameter along its axial length but that is not essential.

Advantageously the shape of the heating element provides a plurality of openings which allow passage of the liquid (and/or produced vapour) therethrough from the wicking element. In the case of the open helical coil outlined above, such openings are provided by the spaces between the turns, but of course other shapes and configurations of openings may be provided depending upon the structure of the heating element. Such openings should ideally be sized so that the beads do not pass through them but so that the beads may partially lodge in them to provide the desirable conformity of the fused wicking element structure with the heating element. Accordingly, in a set of embodiments the characteristic dimension of the openings will be approximately the same order of magnitude as the diameter of the beads. In another set of embodiments, the minimum dimension of the openings may be between 10% and 500%, preferably between 50% and 400%, e.g. between 100% and 300%, of the minimum diameter of the amorphous beads.

The beads which are fused to form the wicking element may be made from any suitable amorphous solid. As used herein, the term “amorphous” is intended to define any substantially non-crystalline material which exhibits a glass transition temperature. Amorphous solids do not melt at a defined temperature but soften and become more fluid gradually as they increase in temperature—this is known as the glass-liquid transition or “glass transition”. The “glass transition temperature” of a material characterises the range of temperatures over which this glass transition occurs. The Applicant has recognised that the use of an amorphous material provides advantages over conventional wicks, such as those made from fused metal balls. Amorphous materials are electrically insulating therefore avoiding the need for any electrically insulated coating to be used in conjunction with a heating element. They are also thermally insulating which minimises the loss of heat from the heating element. The use of an amorphous solid to produce the wicking element provides further advantages such as resistance to fracturing and disintegration during manufacture and in use, and the capability to withstand the temperatures involved in vaporisation of the liquid. Fused beads of an amorphous solid thus provide the desired combination of structural integrity, wicking properties, insulation and liquid hold-up to form the wicking element.

Suitable amorphous solids for use in the invention may be selected by those skilled in the art. When provided in the form of small beads and heated to a temperature below their melting point, these will be capable of softening and fusing without molten material filling up the pores. On cooling these should be capable of forming a substantially rigid (i.e. self-supporting) structure comprising a network of interconnecting pores. Considerations in the selection of a suitable amorphous material may include its temperature resistance, chemical compatibility with the liquid to be vaporised, and biocompatibility. Suitable materials should preferably be medical grade.

All glasses are at least partially amorphous and may be used to produce the wicking element. Examples of traditional “glass” materials which may be used include fused quartz (also known as “fused-silica glass”), soda-lime-silica glass, borosilicate glass, lead oxide glass, aluminosilicate glass and germanium oxide glass. Medical grade glass is readily available. Medical grade glasses, such as borosilicate glass and aluminosilicate glass, are particularly suitable for use in the invention.

Amorphous polymers may also be used to produce the wicking element. Suitable polymeric materials include, but are not limited to, the following: polyetheretherketone (PEEK), polystyrene, polyvinylchloride (PVC), poly methylmethacrylate (PMMA), styrene acrylonitrile (SAN), cyclic olefin copolymer (COC), polycarbonate, polyimide, and combinations of any of these.

As will be understood, any amorphous material selected for use in the device should have a glass transition temperature below the vaporisation temperature of the liquid to be vaporised. The vaporisation temperature of the liquid will vary depending on its constituents, for example whether this comprises water as a carrier for the active components, or an organic solvent such as propylene glycol or glycerol. Any silica glass materials would be suitable for use with any liquid formulation for vaporisation. For a water-based formulation, many types of amorphous polymer would be suitable.

For use with an organic solvent-based formulation, a high temperature polymer may need to be used depending on the vaporisation temperature of the formulation. Suitable materials may be selected accordingly.

The beads of the amorphous solid are fused together to provide a substantially rigid structure comprising a network of interconnecting pores (“voids”). The precise size and shape of the beads may be varied and properties of the wicking element, such as liquid flow rate, wicking properties, capillary action, etc., may be adjusted by appropriate selection of their size and shape. The term “bead” as used herein is intended to refer to pieces of any shape which can be fused together to provide the desired porous structure. It includes spheres, distorted spheres (e.g. prolate spheres), rods, granules, prismatic shapes, etc. Advantageously, the beads will be substantially spherical in shape. The surface of each bead will generally be substantially smooth but that is not essential.

Each bead will generally be up to about 1 mm in diameter (where the diameter is considered to be the maximum diameter in the case where the bead is not spherical). For example, each bead may range from about 100 μm to about 1 mm in diameter.

In an embodiment, these may range from about 200 μm to about 800 μm in diameter. In other embodiments, these may range from about 250 μm to about 600 μm in diameter, or from about 300 μm to about 500 μm in diameter.

The pores created in the wicking element are capable of transporting the liquid to be vaporised by capillary action. Pore sizes can be suitably adjusted to provide effective transport of liquid through the wicking material and controlled delivery. The term “pore size” is used to describe the maximum diameter of the pores of the wicking material. In one embodiment, the pores will be substantially uniform, i.e. the same geometry and pore size. Typically pore sizes should not vary by more than about 50%, preferably not more than about 30%, e.g. not more than about 10%, as this may affect the uniformity of liquid transfer through the material.

The wicking element may have an overall porosity in the range of 5 to 95% by volume, e.g. from about 26% to about 48% by volume. High porosities may be achieved by replacing a proportion of the amorphous solid beads with beads (e.g. spheres) composed of a different material that can be removed after the structure is fused. For example, glass beads may be mixed with calcium carbonate beads, the glass beads are fused and then the calcium carbonate beads are removed (e.g. by dissolving in acid) to leave a porous structure having a high degree of porosity. Low porosities can be achieved using dissimilarly sized beads of an amorphous solid which allow for closer packing and minimise the pore sizes.

In one embodiment, the wicking element is constructed from substantially uniform spheres of an amorphous solid. The precise shape and size of the pores depends on the size of the spheres and the mode of packing. In one embodiment, the spheres may be regularly packed and the resulting pores will be substantially concave in shape.

The device comprises a heating element which is capable of heating the liquid to an appropriate vaporisation temperature in order to produce a condensation aerosol for inhalation by the user. In one set of embodiments, the heating element is formed of a material which provides resistive heating when an electrical current is passed through it. Electrically conductive materials which may be used as resistive heating elements should be thermally stable and chemically non-reactive with the liquid to be heated so that they do not adversely affect the nature of the liquid (e.g. cause degradation of any active components) and do not affect the flavouring (where this contains any flavouring agents). The heating element may comprise any material that becomes heated when an electrical current is passed through it and suitable materials may be selected accordingly. Examples of materials which may be used include nickel-chromium, iron-chromium, aluminium, stainless steel, and titanium. Nickel-chrome is particularly suitable.

As outlined above, the heating element may be provided in a variety of different forms and configurations capable of providing an integrally formed heater-wick element as herein described. For example, it may be provided in the form of fibres, wires, ribbons, spirals, strips, coils, meshes, etc. Conveniently it may be provided in the form of a coil or a mesh, e.g. a wire coil.

In aspects of the invention the device comprises an integrally formed heater-wick element. This may be produced by close packing of the beads which form the wicking element in and/or around a heating element in a suitable mould, fusing the beads together by heating, then removing the resulting heater-wick element from the mould.

As part of the fusing process, the heating element can thus be ‘over-moulded’ into the desired shape. Once fused, the heating element effectively becomes part of the resulting structure, i.e. it is “integrally formed” with the wicking element. The Applicant has recognised that this method of manufacture allows for good thermal contact and structural integrity of the heating element.

Packing of the beads in the mould may be achieved by any suitable means, for example, by gravity or suitable vibration means.

The process for fusing of the beads may be carried out by heating the beads to their glass transition temperature. Where the amorphous material is characterised in having a glass transition temperature which spans a range of temperatures, the beads may be heated to any temperature within that range. As will be understood, heating should be carried out below the melting temperature of the amorphous material to ensure this does not melt. For glasses this temperature is usually between 677° C. and 732° C. The duration of heating will depend on the nature of the amorphous material, size of beads, etc., but can readily be determined by the skilled person. Heat treatment times may be expected to be in the range from 5 to 30 mins, e.g. 10 to 15 mins. The heating process causes partial softening and thus fusing of the beads. The beads do not melt so do not become molten thus ensuring that these essentially retain their original shape during the fusing process. In this way, voids are retained between the beads allowing the resulting wicking element to hold-up and transport a liquid by capillary action.

Various shaped moulds may be used in the production of the heater-wick element thereby providing the ability to mould the combined wick and heating elements into various shapes, as desired. Typically, the moulded wicking element will be cylindrical in shape, e.g. having a substantially uniform diameter along its length, but this is not essential.

In one embodiment, the heating element may be provided around at least a portion of the wick, e.g. so that the wicking element is provided in a radially inner or concave region defined by the heating element. In this “male” arrangement the wicking element may be provided in the form of a cylinder of fused beads, e.g. having a diameter in the range of from about 1 mm to about 10 mm, e.g. from about 3 mm to about 7 mm. The length of the wicking element may be in the range from about 5 mm to about 30 mm. e.g. from about 10 mm to about 20 mm.

In another embodiment, the heating element may be provided within at least a portion of a hollow cavity or concave region defined by the wicking structure. In this “female” arrangement, the cavity should be open to allow the escape of vapour at one or both ends of the wicking element. In this embodiment, the external diameter of the wicking element may be in the range of from about 10 mm to about 30 mm, e.g. from about 15 mm to about 25 mm. The length of the wicking element may be in the range from about 5 mm to 20 mm, e.g. from about 10 mm to about 15 mm.

In another embodiment, the heating element may be provided within at least a portion of a hollow cavity or concave region defined by the wicking structure in which the cavity or region is open to allow the escape of vapour at both ends and part of its side. In this “cutaway” arrangement, the wicking element has a C-shaped cross-section. In this embodiment, the external diameter of the wicking element may be in the range of from about 5 mm to about 30 mm, e.g. from about 15 mm to about 25 mm. The length of the wicking element may be in the range from about 5 mm to 20 mm, e.g. from about 10 mm to about 15 mm. The extent of the cavity opening may be up to about a 180° subtended angle at the central axis of the heater-wick element.

The reservoir may be adapted to hold any liquid which is suitable for vaporisation. The reservoir may be made of any material which is chemically and biologically compatible.

The reservoir used in the device for storing the liquid for aerosolization may take a variety of forms. For example, it may comprise a porous substrate impregnated with the liquid. Porous substrates may include foams or fibrous materials capable of absorbing and retaining the liquid. The liquid may, alternatively, be provided within a container, e.g. a bottle.

The precise configuration (shape, size, etc.) of the reservoir and its engagement with the wicking element may vary provided the reservoir and wicking element are in liquid communication with one another when the device is in operation. Where the reservoir is a porous substrate capable of carrying the liquid, the substrate may engage with the wicking element in any orientation in which at least one face of the substrate is in intimate contact with the wicking element. Any reservoir configurations known and used in the art may be employed in the invention.

In certain embodiments, the reservoir may be provided external to the housing, although more typically it will be contained within the housing. Where it is provided external to the housing, the reservoir may, for example, be a container (e.g. a bottle) and a suitable conduit (e.g. tube or pipe) may be provided for transportation of the liquid from the reservoir to the wicking element.

In use, the wicking element provides for the transport of at least a portion of the liquid from the reservoir to the heating element by capillary action. In applications where a high rate of aerosol production is required or where capillary action is insufficient to supply the liquid to the heating element at the desired rate, additional means may be provided to force the liquid into the heater-wick element such as, but not limited to, any of the following: gravity, pressurised reservoir, mechanical pump, electrical pump, solenoid pump, piezoelectric pump, positive displacement pump or syringe pump. In one set of embodiments, the device may thus further comprise a pump configured to pump liquid from the reservoir through the wicking element. Any pump known and used in the art may be used. As will be understood, any pumping means must be suitable for the viscosity of the liquid formulation and should be selected accordingly. This selection is straightforward for those skilled in the art.

The device may further comprise an electrical power source in electrical connection with the heating element. The electrical power source may be a battery, a capacitor, or a combination thereof.

The heating element is preferably a resistive heating element but this is not essential.

Other heating technologies could be employed as appropriate, such as a Peltier device, micro-mechanical heat pump, combustion of propane/butane, or the like.

The device may further comprise one or more control components that actuate or control the flow of current from the electrical power source to the heating element. The power source and control components will advantageously be provided within the housing thereby providing a compact delivery device which can be hand-held.

The control components may include a switch which can be linked to a control circuit for manual control of power. The switch may be used to turn on the device and/or to actuate the flow of current to the heating element and thus the generation of heat and the desired condensation aerosol. The switch may take any suitable form, such as a pushbutton, a slide switch, a toggle switch, etc. Other control components may be provided, such as those which may be responsive to the user's drawing on the device.

Control components can be configured such that these provide close control over the amount of heat provided by the heating element. In some embodiments, a current regulating component can be provided which can function to stop the flow of current to the heating element once a predetermined temperature has been reached. Such a predetermined temperature may be one which is sufficient to volatilise the liquid and provide a required amount of aerosol for one draw (or puff) by the user.

The power source should preferably be capable of delivering sufficient power to rapidly heat the heating element to provide the desired aerosol. Suitable power sources include lithium ion batteries (e.g. rechargeable lithium ion batteries) however other types of batteries may be used. Where rechargeable batteries are used, the device may additionally comprise charging contacts for connection to a corresponding contact in a recharging unit. In other embodiments, the power source may comprise a capacitor.

The heat required to volatilise the liquid and in a sufficient amount for a single draw or puff on the device will vary depending on the nature of the liquid and the desired volume of the draw. However, typically, the heating element may be heated to a temperature in the range of about 100° C. or higher, e.g. about 150° C. or higher, or about 200° C. or higher, e.g. to a temperature in the range of from about 150° C. to about 250° C. The temperature and duration of heating can be controlled as described herein.

Energy to the heating element may be controlled, for example by delivering constant power or constant temperature. Temperature can either be independently measured or derived from the resistance of the heater element.

In one embodiment, the device further comprises a temperature sensor. The temperature sensor may be thermally coupled to the heating element in order to determine the temperature of the heating element. The temperature sensor may be integrated into control circuitry which monitors the temperature of the heating element using the temperature sensor and then controls the heating element based on the measured temperature. The control circuitry may take the form of a printed circuit board (PCB)

In one embodiment, the control circuitry delivers constant power to the heating element. The power may be provided via a pulse width modulated (PWM) signal with the power parameters derived from the measuring supply voltage.

In one embodiment, the heating element comprises a resistance heater and a second resistive element. In this embodiment the heating element may be adapted to have a known relationship between the temperature and electrical resistance with the second resistive element. The heating element may have a first configuration in which the resistance heater may generate heat, and wherein the resistance heater and the second resistive element are effectively not in series with one another; and a second configuration in which a temperature measurement is made, wherein the resistance heater and the second resistive element are arranged in series with each other such that a current passing through the resistance heater is reduced compared to a current passing through the resistance heater when heating in the first configuration. The device may be arranged in said second configuration to take a measurement to determine the electrical resistance of the heater thereby allowing the temperature of the resistance heater to be determined using the known relationship between its temperature and electrical resistance.

The device includes an airflow path through the device such that the aerosol generated can be withdrawn from the device by a user drawing on the device. The specific positioning of the components within the device can vary provided that, in use, the heat from the heating element can volatilise the liquid drawn from the reservoir by the wicking element and form an aerosol for inhalation by the user.

The device further comprises an outlet to allow exit from the housing of a condensation aerosol formed by the vaporised liquid and at least one air inlet which is provided in an external wall of the housing. The air inlet is arranged to provide an air path from the outside of the device to the heating element (where it contacts the generated aerosol) and from the heating element to the outlet. The device will generally comprise a mouthpiece which is in communication with the outlet and can provide for passage of the air and generated aerosol from the heating element to the user's mouth.

In one embodiment, the air inlet and outlet are arranged such that the passage of air carrying the aerosol to the user is drawn axially along the length of the aerosol delivery device. In this embodiment the air flow will typically pass over the control electronics and power source.

In an alternative embodiment, the air inlet and heater-wick element may be arranged such that the air does not have to flow along the length of the device. For example, the air inlet may be arranged such that air is drawn from the outside of the device directly to the heating element without passing over any control circuitry or power source. Such an arrangement has the advantage that if excess liquid is drawn by the wicking element and is not vaporised, this is prevented from flowing down the air path into the electronics and battery or other power source. Any liquid flow into the control electronics and/or power source could give rise to the need to provide extra sealing in these areas of the device which may add to cost. In this embodiment, the flexibility in manufacturing of the heater-wick element as herein described provides for the possibility of alternative geometries of the heater-wick element. Such geometries may, for example, provide for the option of a side flow of air to pass from the exterior of the device to the heating element, for example in the case where the wicking element has a cutaway portion which exposes at least a portion of the heating element to the side flow of air (e.g. the “cutaway” arrangement described above).

In use, inhalation by the user causes air to flow from the outside of the device to the heating element whereafter the aerosol is produced which then exits the outlet and passes to the user's mouth and lungs.

Different users inhale at different flow rates. In certain embodiments, it may therefore be advantageous to split the air flow through the device to provide a constant air flow across the heating element irrespective of how hard the user inhales. The particle size of the aerosol can also be adjusted by varying the air flow over the heating element. This enables adjustment of the particle size of the aerosol which is inhaled by the user. In an embodiment the device may thus further comprise a separate air flow path (i.e. by-pass air flow path) arranged such that only a portion of the incoming air flow passes over the heating element. For example, the by-pass air flow path may be provided by a split in a flow path of incoming air upstream of the heating element whereby a selected portion of the air flow passes to the heater-wick element. Downstream of the heating element the separate air flow paths may recombine so that both air flows pass to the outlet and to the user. Alternatively, these may be maintained separately and pass to separate outlets in the device. In this way, only the air flow containing the condensation aerosol is inhaled by the user. The remaining air flow exits the device to the surrounding air.

Where the device contains a “by-pass” air flow path, this may include suitable control means to control the flow of air into the by-pass air flow path depending on the rate of inhalation by the user. Suitable control means may include an adjustable valve or flap, e.g. a valve or flap which is activated by differential pressure. In such embodiments, the harder the user inhales on the device, the greater the air flow channelled down the by-pass air flow path. The use of a suitable “by-pass” enables a constant air flow past the heating element irrespective of the flow generated by the user.

In the same way that it is advantageous to keep the air flow across the heating element constant when a user draws on the device, i.e. during an inhalation, it may also be advantageous to adjust the air inlet and/or air outlet temperature to maintain consistent performance of the device and/or to modify the particle size of the aerosol which is generated. In one set of embodiments, an additional heater such as a second electrically powered heating element may be provided in series either before (i.e. upstream) or after (i.e. downstream) of the described heater-wick element whereby to control the temperature of the air flow.

The device may comprise first and second parts which are engageable and disengageable with one another. In one set of embodiments, the heater-wick element and the electrical power source can be removably connected. For example, a first part of the device may comprise the heater-wick element and the second part may comprise the electrical power source (the “control body”). The first part will also typically contain the reservoir. The second part will also typically contain the control components that actuate or control the flow of current from the electrical power source. The first part may be disposable.

The liquid for use in the device may contain any combination of components which are suitable for aerosolization. The vaporising liquid preferably contains a carrier liquid and an active drug. The carrier liquid may be any conventional carrier liquid which is chemically and biologically compatible with the active drug. Suitable examples of carrier liquids include, but are not limited to, propylene glycol, methanol, ethanol, dichloromethane, methyl ethyl ketone, diethyl ether, glycerol, and dimethylformamide.

When used as an e-cigarette, the liquid may include tobacco, a tobacco component, or a tobacco-derived material such as nicotine. It may also contain one or more flavourings. Flavouring agents may be natural or artificial and can include any flavorings traditionally used for the flavouring in cigarette, cigar or pipe tobaccos, e.g. fruit flavours, menthol, mint, peppermint, cocoa, licorice, cinnamon, etc.

Active ingredients may include respiratory drugs such as asthma drugs, chronic obstructive pulmonary disease drugs, pulmonary hypertension drugs, pulmonary fibrosis drugs, or cystic fibrosis drugs.

Classes of bronchodilator drugs suitable for use with the described methods and devices include the β-adrenergics, the methylxanthines, and the anticholinergics.

Classes of anti-inflammatory drugs suitable for use with the described methods and devices include the corticosteroids, the mediator-release inhibitors, the anti-leukotriene drugs, as well as other inhibitors or antagonists.

Other classes of respiratory drugs suitable for use with the described methods and devices include anti-endothelin drugs and prostacyclin drugs, which are particularly useful in the treatment of pulmonary fibrosis or hypertension, and ion channel or pump inhibitors, enhancers, and modulators, which are particularly useful in the treatment of cystic fibrosis.

Examples of β-adrenergics include albuterol, epinephrine, metaproterenol, terbutaline, pseudoephedrine hydrochloride, bambuterol, bitolterol, carbuterol, clenbuterol, clorprenalin, dioxethedrine, eprozinol, etefedrine, ethylnorepinephrine, fenoterol, fenspiride, hexoprenaline, isoetharine, isoproterenol, mabuterol, methoxyphenamine, pirbuterol, procaterol, protokylol, rimiterol, salmeterol, soterenol, tretoquinol, tulobuterol, and pharmaceutically acceptable salts and mixtures thereof. Examples of methylxanthines include caffeine, theophylline, aminophylline, acefylline, bamifylline, doxofylline, dyphylline, etamiphyllin, etofylline, proxyphylline, reproterol, theobromine-1-acetic acid, and pharmaceutically acceptable salts and mixtures thereof. Examples of anticholinergics include atropine, ipratropium bromide, flutropium bromide, oxitropium bromide, tiotropium bromide, and pharmaceutically acceptable salts and mixtures thereof. Examples of corticosteroids include budesonide, beclomethasone, ciclesonide, dexamethasone, flunisolide, fluticasone propionate, triamcinolone acetonide, prednisolone, methylprednisolone, hydrocortisone, and pharmaceutically acceptable salts and mixtures thereof. Examples of mediator-release inhibitors include cromolyn sodium, nedocromil sodium, and pharmaceutically acceptable salts and mixtures thereof. Examples of anti-leukotrienes include montelukast, zafirlukast, and pharmaceutically acceptable salts and mixtures thereof.

Other suitable respiratory drugs include pirfenidone, CPX, IBMX, cilomilast, roflumilast, pumafentrine, domitroban, israpafant, ramatroban, seratrodast, tiaramide, zileuton, ambrisentan, bosentan, enrasentan, sitaxsentan, tezosentan, iloprost, treprostinil, and pharmaceutically acceptable salts and mixtures thereof.

In other embodiments the active drug may be selected from the group consisting of aclidinium bromide, alprazolam, clonazepam, fentanyl, fluphenazine hydrochloride, formoterol, glcopyrrolate, haloperidol, Ilioperidone, indacaterol, mometasone, olanzapine, olodaterol, risperidone, trifluoperazine, umeclidinium bromide, and zolmitriptan, and pharmaceutically acceptable salts thereof.

As will be understood, the drug should be heat stable.

In some embodiments the vaporising liquid may contain at least 50% by weight of an active ingredient, for example from 60 to 95%, or from 70 to 90%, or from 75 to 80% by weight of the active ingredient. In other embodiments, for example when using the device to deliver an aerosol for use in an e-cigarette, the amount of active ingredients may be lower and may comprise from 0.5 to 10% by weight of the liquid, for example up to 8% by weight, or up to 5% by weight.

The amount of liquid present in the device will be dependent on various factors, such as the nature of the device and the intended action of the liquid, the number of draws (puffs) intended for each reservoir, the desired volume of each draw (puff), etc. Typically, the amount of liquid may be less than about 1.5 g, e.g. less than about 1.0 g.

In a further aspect the invention provides a kit for delivering a drug condensation aerosol comprising: (a) a composition comprising one or more active components (e.g. a drug, or flavouring agent), preferably in unit dose form; and (b) a device for forming a drug condensation aerosol as herein descried. The composition will further comprise one or more known pharmaceutically acceptable carriers or excipients. These may be volatile or non-volatile.

Methods of treating a disorder, such as a respiratory disorder, using the aerosol delivery device herein described also form part of the invention. Such methods comprise the step of administering to a patient in need thereof a therapeutically effective amount of a drug condensation aerosol using an aerosol delivery device as herein described. A “therapeutically effective amount” is the amount required to achieve the desired therapeutic effect and includes prevention. Disorders which may be treated include respiratory disorders or diseases, such as asthma, chronic obstructive pulmonary disease, pulmonary hypertension, pulmonary fibrosis, and cystic fibrosis.

The method for manufacturing the heater-wick element herein described allows for the integral incorporation of the heating element into the heater-wick element and/or for the formation of the wicking element so as to at least partially conform to the shape of the heating element, for example by over-moulding a coiled heating element, which increases product performance. By moulding the heater-wick element in this way, it can be more easily manufactured, e.g. produced by machine rather than by hand. The process thus lends itself to medical manufacture.

Therefore the heater-wick element herein described and the method for its manufacture form further aspects of the invention.

Viewed from a yet further aspect the invention thus provides a heater-wick element which comprises:
a heating element; and
a wicking element formed from fused beads of an amorphous solid which provides a porous structure adapted to transport a liquid for vaporisation to the heating element;
wherein the heating element is integrally formed with, or formed so as to at least partially conform to a shape of, the wicking element and is operable to heat the wicking element thereby vaporising at least a portion of the liquid transported by the wicking element to allow a condensation aerosol to be formed.

Viewed from a further aspect the invention provides a method of manufacturing a heater-wick element, said method comprising:
providing a heating element in a mould;
packing a plurality of beads of an amorphous solid in said mould and in intimate contact with said heating element;
fusing said beads to provide a substantially rigid structure; and
removing the resulting heater-wick element from the mould.

Although the invention has been described primarily in the context of an aerosol delivery device for personal use, such as an e-cigarette, it will be understood that the device is not limited to such a purpose or shape.

The wick and heating element arrangement described herein may be used in other vapour-dispensing devices in which a fluid is heated with a heating element to produce a vapour, e.g. a vapour for delivery to the surrounding air, e.g. for the delivery of fragrance vapours, perfumes, etc. Examples of such devices include, but are not limited to, any of the following: electric liquid air fresheners, scent delivery systems, fumigation devices, and humidifiers. Types of liquids which may be vaporised include oily liquids such as volatile fragrance substances, e.g. essential oils and aromatic chemicals.

In a further aspect the invention thus provides a vaporiser for the generation of a vapour from a liquid, said vaporiser comprising:
a reservoir adapted to contain a liquid for vaporisation;
a heating element having a pre-defined shape;
a wicking element formed from fused beads of an amorphous solid so as at least partially to conform to the shape of the heating element, thereby providing a porous structure adapted to transport liquid from the reservoir to the heating element such that the heating element is operable to heat the wicking element thereby vaporising at least a portion of the liquid transported from the reservoir by the wicking element; and
an airflow path to allow the flow of a condensation aerosol formed by said vaporised liquid.

In another aspect the invention provides a vaporiser for the generation of a vapour from a liquid, said vaporiser comprising:
a reservoir adapted to contain a liquid for vaporisation;
a heating element;
a wicking element formed from fused beads of an amorphous solid and integrally formed with the heating element, thereby providing a porous structure adapted to transport liquid from the reservoir to the heating element such that the heating element is operable to heat the wicking element thereby vaporising at least a portion of the liquid transported from the reservoir by the wicking element; and
an airflow path to allow the flow of a condensation aerosol formed by said vaporised liquid.

Where appropriate, further embodiments of the vaporiser according to the invention may include any of the features herein described in respect the aerosol delivery device.

A number of embodiments of the invention will now be described, by way of example only, with reference to the accompanying drawings in which:

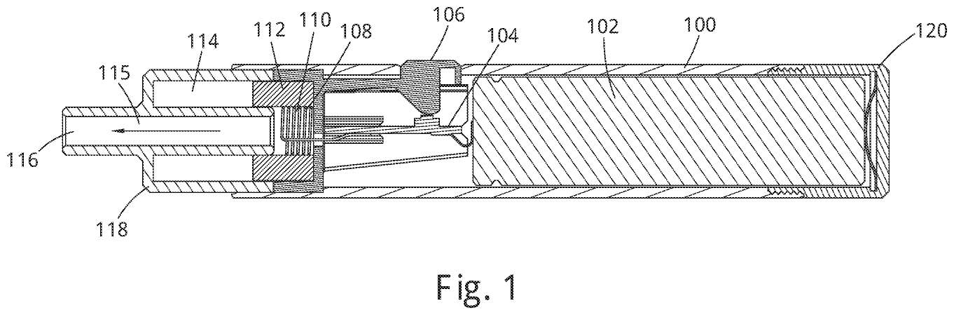

FIG. 1 is a cross-section of an aerosol delivery device in accordance with the present invention;

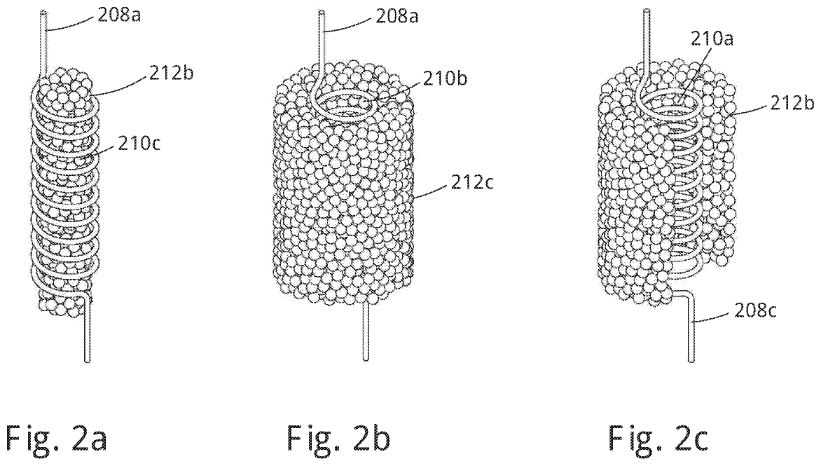

FIGS. 2a, 2b and 2c illustrate heater-wick elements in accordance with the present invention;

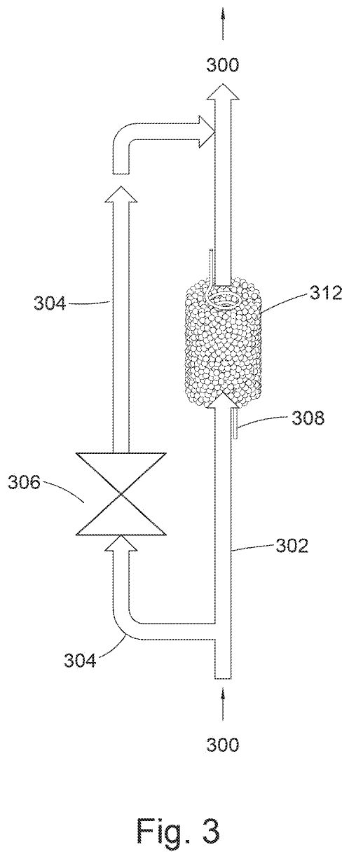

FIG. 3 is a schematic illustration of a by-pass air flow path arranged to control air flow across a heater-wick element in accordance with the present invention; and

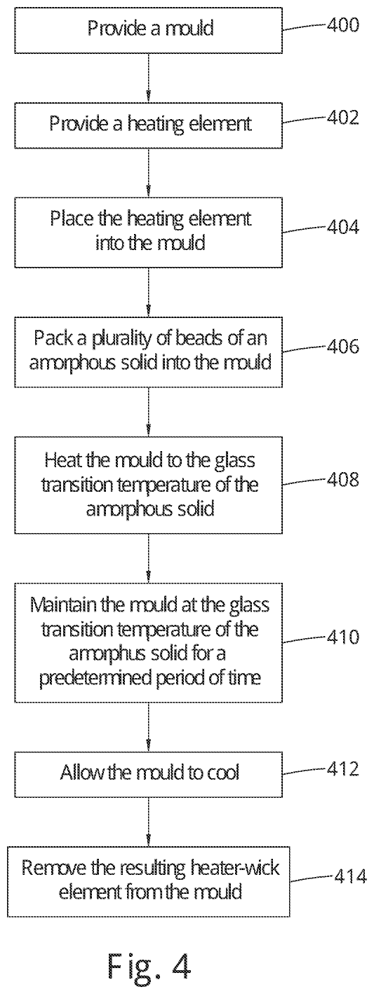

FIG. 4 is a flow chart showing a method for producing a heater-wick element in accordance with the present invention.

FIG. 1 shows a cross-section of an aerosol delivery device in accordance with an embodiment of the invention. The device is an electronic inhaler which comprises a housing 100, 118 containing the various components of the device. In this embodiment the housing is generally cylindrical in shape and comprises two separable parts, a first part 118 and a second part 100. The first and second parts of the housing may be made from various materials, including metals, non-metals (e.g. plastics such as polypropylene, PEEK or polyethylene) and composite materials. Where the second part of the housing 100 is made from a metal, a common earth may be provided in the device in order to simplify the design.

Provided within the second part of the housing 100 is a power source 102, e.g. a battery, which is electrically connected to a control component 104. The control component 104 is a printed circuit board (PCB) which has a means of switching energy transfer to the heating element 108. The control component 104 is electrically connected to a switch 106, for example a pushbutton which extends to an exterior surface of the second part of the housing 100.

The second part of the housing 100 contains a heating element 108 provided in the form of a helical coiled wire which is electrically connected to the control component 104. The heating element 108 comprises a plurality of openings 110 formed between the spaces of the turns of the coil. In this embodiment the heating element 108 is ‘over-moulded’ by a cylindrical wicking element 112 which is formed from fused amorphous beads.

The first part of the housing 118 includes a reservoir 114 which contains a liquid for aerosolization. In reservoir 114 the liquid may be stored as a free liquid or retained within a suitable porous (e.g. reticulated foam) structure. Wicking element 112 is in liquid communication with the reservoir 114. An outlet 116 in the first part of the housing 118 provides an opening extending from the heating element to the exterior of the housing. In this embodiment, outlet 116 is shaped in the form of a tube which may function as a suitable mouthpiece.

An airflow path 115 is provided within the first part of the housing 118. When in use, the airflow path 115 is configured to allow the flow of a condensation aerosol formed by vaporisation of the liquid out of the device and to the user. The airflow path 115 may extend between one or more air inlets (not shown) provided in an external wall of the housing and the outlet 116 and may take any path through the device.

The housing may be provided in two or more separable parts enabling the replacement of one or more parts of the device. For example, the first part 118 may be separable from the second part 100 enabling replacement of the first part when the liquid for vaporisation has been depleted from the reservoir 114. Alternatively, the first part may be removed and the reservoir 114 may be re-filled with liquid.

The first part of the housing 118 is attached to the second part of the housing 100 by a click connection. In this embodiment one end of the second part of the housing 100 is attached by a screw thread allowing for easy removal of the power source 102. The power source 102 may be removed and either replaced or recharged.

Operation of the device will now be described with reference to FIG. 1. In use, a user draws on the outlet 116 of the device. This draws air from an inlet in the housing (not shown) and into contact with the heating element 108. The air then passes to the outlet 116. Whilst drawing on the device, the user switches on the device by depressing the pushbutton 106. This completes the electrical connection between the power source 102 and control component 104. When switched on, the control component 104 directs an electrical current through the heating element 108 causing it to heat up. The wicking element 110 simultaneously functions to supply the liquid (containing one or more active ingredients) from the reservoir 114 to the heating element 108 by capillary action. When the liquid comes into contact with the heating element 108 it vaporises. The vaporised liquid is drawn away from the heating element 108 and towards the outlet 116. As the vapour cools it forms a condensation aerosol which is inhaled by the user.

The control component 104 monitors the temperature of the heating element 108. For example, the temperature of the heating element 108 can be monitored by using the known relationship between the resistance of the heating element 108 and its temperature. If the heating element 108 becomes too hot the control component 104 stops the electrical current supply to the heating element 108, which in turns allows the heating element 108 to cool down. When the temperature of the heating element 108 falls below a pre-set temperature required for vaporisation of the liquid, control component 104 re-activates the electrical current supply to the heating element 108 thereby increasing its temperature. In this way the temperature of the heating element 108 can be suitably controlled during use of the device according to the chosen liquid and its vaporisation temperature.

At any point, the user can stop production of the vapour (and thus the condensation aerosol) by releasing the pushbutton 106. This terminates the electrical connection between the power source 102 and control component 104.

FIG. 2 shows various embodiments of the heater-wick element in accordance with the present invention.

In FIG. 2a a heating element 208a is provided in the form of an open helical coil of wire arranged to provide a plurality of openings 210a between the individual turns of the coil. The heating element 208a conforms to the shape of the wicking element 212a which is formed from fused amorphous beads and which is provided in a radially inner or concave region defined by the heating element 208a. The openings 210a in the heating element 208a are sized such that the beads of the wicking element 212a do not pass through them but partially lodge in them to form the desired conformity of the fused wicking element 212a with the heating element 208a. The embodiment shown in FIG. 2a is an example of the “male” arrangement herein described.

FIG. 2b shows an alternative arrangement of the wicking element 212b and the heating element 208b. In this embodiment the heating element 208b is provided in the form of an open helical coil of wire arranged to provide a plurality of openings 210b between the individual turns of the coil. The heating element 208b is provided within a concave region defined by the wicking element 212b. The openings 210b in the heating element 208b are sized such that the beads of the wicking element do not pass through them but partially lodge in them to form the desired conformity of the fused wicking element 212b with the heating element 208b. In this arrangement the inner region defined by the heating element 210b is open to allow the escape of vapour at both ends of the wicking element 212b. The embodiment shown in FIG. 2b is an example of the “female” arrangement herein described.

FIG. 2c shows an alternative arrangement of the wicking element 212c and the heating element 208c. In this embodiment the heating element 208c is provided in the form of an open helical coil of wire arranged to provide a plurality of openings 210c between the individual turns of the coil. The heating element 208c is provided within a partially concave region defined by the wicking element 212c. In this embodiment, the wicking element 212c does not completely surround the heating element 208c but provides a cut-away portion. This allows for incoming air flow from the side of the heating element 208c. The embodiment shown in FIG. 2c is an example of the “cut-away” arrangement herein described.

The heater-wick element in FIG. 2a may be manufactured by providing a hollow cylindrical shaped mould made from a material capable of withstanding the glass transition temperature of the amorphous beads which make up the wicking element 212a. A pre-formed heating element 208a is positioned within the mould. Amorphous beads are then placed into the radially inner region defined by the heating element 208a so that they come into contact with the inner region of the heating element 208a, but cannot pass through the openings 210a between the turns of the coils. The mould containing the amorphous beads and heating element 208a is then heated to the glass transition temperature of the amorphous material. This causes the beads to soften and fuse with one another. The resulting “integral” heater-wick element is then removed from the mould, once cooled.

FIG. 3 is a schematic illustration of a by-pass air flow path arranged to control air flow across a heater-wick element in accordance with the present invention. Incoming air flow 300 is split into two separate air streams which follow separate air flow paths 302, 304. Air flow path 302 provides a constant flow of air which passes through the heater-wick element comprising a heating element 308 and wicking element 312. Air flow path 304 (the “by-pass” airflow path) is diverted from the heater-wick element and passes through a suitable control means 306, for example a valve, adapted to control the flow of air. In this embodiment, the separate air flows 302, 304 combine downstream of the heater-wick element whereafter they pass to the user. When the device is in use the “by-pass” airflow path enables a constant and controllable air flow past the heating element irrespective of the air flow generated by the user when drawing on the device.

FIG. 4 illustrates a method for producing a heater-wick element in accordance with an embodiment of the invention. The method described below is intended to be illustrative only and thus non-limiting. In some embodiments, the method may be accomplished using one or more additional steps which are not shown.

At step 400, a mould is provided. The mould may be any shape or size suitable for forming a heater-wick element as herein described. Typically, it will be cylindrical in shape. The mould can be made of any material capable of withstanding heating to the glass transition temperature of the amorphous solid which is to be used for forming the wicking element. In one embodiment the mould can be a cylindrical graphite mould. At step 402, a suitable heating element (e.g. a wire coil) having a configuration appropriate for insertion into the mould is provided. At step 404, the heating element is inserted into the mould. Depending on the desired configuration of the heater-wick element, the heating element may be provided in intimate contact with the internal surface of the mould, or it may be mounted in the centre of the mould providing a hollow cavity or concave region defined by the outer surface of the heating element and the internal surface of the mould. In the case where the heating element is not in intimate contact with the internal surfaces of the mould, a suitable support may be provided to hold the heating element in position during the formation of the heater-wick element. At step 406, a plurality of beads of an amorphous solid are packed into the mould, e.g. by pouring these into the mould cavity formed within or around the heating element. The beads may be randomly or non-randomly packed. Non-random or uniform packing may improve the uniformity of the pores formed between the beads. The beads are packed into the mould such that these form an intimate engagement with the heating element. At step 408, the mould containing the packed beads and the heating element is heated to the glass transition temperature of the amorphous material. Heating may be carried out in a single step or it may comprise multiple heating steps. Heat may be provided by any means known in the art, for example by placing the mould and its contents in a kiln. At step 410, the mould is held at the glass transition temperature of the amorphous material for a defined period of time. This should be sufficient for the beads to soften and fuse together and will be dependent on the nature of the amorphous solid. At step 412, the mould is cooled. The cooling step may be performed over any suitable period of time and may include one or more cooling stages. The rate of cooling may be increased by the use of suitable cooling means known in the art. At step 414, the resulting “integrally formed” heater-wick element is removed from the mould.

The invention is illustrated further by way of the following non-limiting example:

### EXAMPLE

A heater-wick element in accordance with the invention is made as follows:

A wire heater element is wound on a threaded bar to produce an open helical coil. The threaded bar is removed and replaced with a smooth graphite rod. The assembly is mounted concentrically inside a cylindrical graphite mould. Borosilicate glass beads (0.3 mm diameter) are poured around the central rod and coil assembly until the mould is filled. The entire assembly is fired in a kiln. It is heated up to 700° C. at a rate of 100° C. per hour. It is held at that temperature for 10 minutes then rapidly cooled to 516° C. where it is held for 2 hours before being slowly cooled to room temperature with the kiln off. Once cool the fused beads are removed from the mould and the central graphite rod is removed. This produces a “female” heater-wick similar to that illustrated in FIG. 2b.

1. An electronic inhaler for the generation of a condensation aerosol from a liquid comprising:
a reservoir adapted to contain a liquid for aerosolization;
a heating element having a pre-defined shape;
a wicking element formed from fused beads of an amorphous solid so as to at least partially conform to the shape of the heating element, thereby providing a porous structure adapted to transport liquid from the reservoir to the heating element such that the heating element is operable to heat the wicking element thereby vaporising at least a portion of the liquid transported from the reservoir by the wicking element; and
an airflow path to allow the flow of a condensation aerosol formed by said vaporised liquid.

2. An electronic inhaler for the generation of a condensation aerosol from a liquid comprising:
a reservoir adapted to contain a liquid for aerosolization;
a heating element;
a wicking element formed from fused beads of an amorphous solid and integrally formed with the heating element, thereby providing a porous structure adapted to transport liquid from the reservoir to the heating element such that the heating element is operable to heat the wicking element thereby vaporising at least a portion of the liquid transported from the reservoir by the wicking element; and
an airflow path to allow the flow of a condensation aerosol formed by said vaporised liquid.

3. An electronic inhaler according to claim 1 or claim 2, wherein the amorphous solid is a glass, preferably wherein the glass is selected from fused quartz, soda-lime-silica glass, borosilicate glass, lead oxide glass, aluminosilicate glass and germanium oxide glass.

4. An electronic inhaler according to claim 1 or claim 2, wherein the amorphous solid is an amorphous polymer, preferably wherein the amorphous polymer is selected from polyetheretherketone, polystyrene, polyvinylchloride, poly methylmethacrylate, styrene acrylonitrile, cyclic olefin copolymer, polycarbonate, polyimide, and any mixtures thereof.

5. An electronic inhaler according to any one of the preceding claims, wherein the wicking element is formed from fused beads of an amorphous solid which are substantially spherical in shape.

6. An electronic inhaler according to any one of the preceding claims, wherein each of the fused beads independently has a diameter ranging from about 100 μm to about 1 mm.

7. An electronic inhaler as claimed in any one of the preceding claims, wherein the wicking element comprises substantially uniform spheres of an amorphous solid.

8. An electronic inhaler according to any one of the preceding claims, wherein the wicking element comprises a plurality of pores having pore sizes which do not vary by more than about 50%.

9. An electronic inhaler according to any one of the preceding claims, wherein the heating element provides a plurality of openings which allow passage of the liquid therethrough from the wicking element.

10. An electronic inhaler according to any one of the preceding claims, wherein the heating element comprises wire.

11. An electronic inhaler according to claim 10, wherein the heating element comprises wire which is formed into an open helical coil shape and the wicking element has an undulating shape which substantially conforms to the contours of the coil.

12. An electronic inhaler according to claim 11, wherein the heating element provides a plurality of openings provided by the spaces between the turns of the coil which are sized so that the beads of the wicking element do not pass through them but may partially lodge in them.

13. An electronic inhaler according to any one of the preceding claims, wherein the heating element is formed of a material which provides resistive heating when an electrical current is passed through it.

14. An electronic inhaler according to any one of the preceding claims, wherein the wicking element is provided in a radially inner or concave region defined by the heating element.

15. An electronic inhaler according to any one of claims 1 to 13, wherein the heating element is provided within at least a portion of a hollow cavity or concave region defined by the wicking structure.

16. An electronic inhaler according to any one of claims 1 to 13, wherein the heating element is provided within at least a portion of a hollow cavity defined by the wicking structure in which the cavity is open to allow the escape of vapour at both ends and part of its side when in use.

17. An electronic inhaler according to claim 16, wherein the extent of the cavity opening is up to about a 180° subtended angle at the central axis of the combined heater-wick element.

18. An electronic inhaler according to any one of the preceding claims, wherein the reservoir comprises a porous substrate capable of impregnation with the liquid.

19. An electronic inhaler according to any one of the preceding claims which further comprises an electrical power source in electrical connection with the heating element.

20. An electronic inhaler according to claim 19 which further comprises at least one control component adapted to actuate or control a flow of current from the electrical power source to the heating element.

21. An electronic inhaler according to claim 20, wherein said control component is a current regulating component configured to stop the flow of current to the heating element once a predetermined temperature has been reached.

22. An electronic inhaler according to any one of the preceding claims which comprises a housing containing said heating element, said wicking element, said airflow path and, optionally, said reservoir.

23. An electronic inhaler according to claim 22 which further comprises at least one air inlet in an external wall of the housing arranged to provide an airflow path from the outside of the housing to the heating element and from the heating element to at least one outlet in an external wall of the housing.

24. An electronic inhaler according to claim 23, wherein the air inlet and outlet are arranged such that the passage of air carrying the formed aerosol to a user is drawn axially along the length of the inhaler when in use.

25. An electronic inhaler according to claim 23, wherein the air inlet and outlet are arranged such that air is drawn from the outside of the inhaler directly to the heating element without passing over any control components or power source when in use.

26. An electronic inhaler according to any one of the preceding claims which is configured to provide a constant air flow across the heating element when in use.

27. An electronic inhaler according to any one of the preceding claims which further comprises a by-pass air flow path arranged such that only a portion of the incoming air flow passes over the heating element when in use.

28. An electronic inhaler according to claim 27, wherein the by-pass air flow path comprises control means to control the flow of air into the by-pass air flow path depending on the rate of inhalation by a user drawing on the inhaler when in use.

29. An electronic inhaler according to any one of the preceding claims which contains a liquid for the generation of said condensation aerosol, wherein said liquid comprises a carrier liquid and an active drug.

30. An electronic inhaler according to claim 29, wherein the carrier liquid is selected from the group consisting of propylene glycol, methanol, ethanol, dichloromethane, methyl ethyl ketone, diethyl ether, glycerol, dimethylformamide and combinations thereof.

31. An electronic inhaler according to claim 29 or claim 30, wherein the active drug comprises tobacco, a tobacco component, or a tobacco-derived material such as nicotine, and optionally one or more flavouring agents.

32. An electronic inhaler according to claim 29 or claim 30, wherein the active drug comprises a respiratory drug, preferably wherein said respiratory drug is selected from asthma drugs, chronic obstructive pulmonary disease drugs, pulmonary hypertension drugs, pulmonary fibrosis drugs, and cystic fibrosis drugs.

33. A kit for delivering a drug condensation aerosol comprising: (a) a liquid composition comprising one or more active components (e.g. an active drug as defined in claim 31 or claim 32); and (b) an electronic inhaler according to any of claims 1 to 28.

34. A heater-wick element which comprises:
a heating element; and
a wicking element formed from fused beads of an amorphous solid which provides a porous structure adapted to transport a liquid for vaporisation to the heating element;
wherein the heating element is integrally formed with, or formed so as at least partially to conform to a shape of, the wicking element and is operable to heat the wicking element thereby vaporising at least a portion of the liquid transported by the wicking element to allow a condensation aerosol to be formed.

35. A heater-wick element according to claim 34, wherein said heating element and/or said wicking element are as defined in any one of claims 1 to 17.

36. A method of manufacturing a heater-wick element according to claim 34 or claim 35, said method comprising:
providing a heating element in a mould;
packing a plurality of beads of an amorphous solid in said mould and in intimate contact with said heating element;
fusing said beads to provide a substantially rigid structure; and
removing the resulting heater-wick element from the mould.

37. A method as claimed in claim 36, wherein the step of fusing said beads of an amorphous solid is effected by heating said beads to the glass transition temperature of the amorphous solid.
